(19) United States
(12) Patent Application Publication
(10) Pub. No.: US 2022/0347352 A1
(43) Pub. Date: Nov. 3, 2022

(54) CELL SUSPENSION COMPOSITION WITH THERAPEUTIC POTENTIAL AND RELATED METHODS AND SYSTEMS FOR IDENTIFYING SAME

(71) Applicant: AVITA Medical, Inc., Valencia, CA (US)

(72) Inventors: Katie BUSH, Marblehead, MA (US); Niraj Kumar DOSHI, Valencia, CA (US); Amelia Ruth HOPKIN, Aliso Viejo, CA (US); Briana Rachel LEE ALTMAN, Mission Viejo, CA (US)

(21) Appl. No.: 17/725,255

(22) Filed: Apr. 20, 2022

Related U.S. Application Data
(63) Continuation of application No. 17/725,222, filed on Apr. 20, 2022.
(60) Provisional application No. 63/182,590, filed on Apr. 30, 2021.

(51) Int. Cl. A61L 27/60; A61P 17/02; A61K 35/36; A61L 27/36

(52) U.S. Cl. CPC A61L 27/60; A61P 17/02; A61K 35/36; A61L 27/362

(57) ABSTRACT

Systems and methods for identifying a cell suspension with therapeutic potential for skin regeneration and related compositions are disclosed herein. In some variations, a method may include receiving a cell suspension that comprises a population of viable cells and non-viable cells, then measuring a value indicative of at least one characteristic of the cell suspension, such as but not limited to one or more of total cell count, total cell viability, cell viability percentage, and median live cell diameter. A cell suspension composition having therapeutic potential may comprise a cell suspension met certain thresholds relating to total cell count, total viable cell count, cell viability percentage, and median live cell diameter.

### RELATED APPLICATIONS

This application is a continuation of U.S. patent application Ser. No. 17/725,222 filed Apr. 20, 2022, which claims the benefit of U.S. Provisional Patent Application No. 63/182,590 filed Apr. 30, 2021, the disclosure of each of which is incorporated herein by reference in its entirety.

### TECHNICAL FIELD

The present invention generally relates to the field of regenerative medicine.

### BACKGROUND

Generally, organ (e.g., skin, etc.) failure and wounds to an organ may be difficult to treat. More particularly, skin wound treatment and wound healing can be a significant problem for some individuals. For example, chronic, non-healing lacerations, scars, or skin openings can adversely affect an individual's health. Additionally, following a trauma causing acute skin wounds, skin may repair itself slowly. Furthermore, some skin diseases or other conditions such as genodermatoses, pigmentation disorders, aging, or other organ failure or insufficiencies, may also benefit from therapeutic interventions to promote healthy or more youthful skin.

One such therapeutic intervention may include preparing a cell suspension containing skin cells and applying the cell suspension over an injured or primed skin site for treatment. When properly prepared and applied, such cell suspensions may offer therapeutic value in promoting skin regeneration for wounds and other applications. However, the therapeutic efficacy of a cell suspension may vary drastically depending on factors such as the quality of the tissue source of the cells and/or the procedure for preparing the cell suspension.

It is challenging to determine the viability of a cell suspension with therapeutic potential for skin regeneration prior to application of the cell suspension. Rather, therapeutic efficacy is known only after prepared cell suspension is applied, as the treated area may be monitored thereafter. Treatment may need to be repeated if it becomes apparent that the cell suspension was not effective. However, repeating the process multiple times extends the period of treatment, and thus may lead to prolonged discomfort or even deteriorating patient health.

Therefore, there is an unmet need for cell suspension compositions having therapeutic potential, as well as methods and systems for identifying a cell suspension with therapeutic potential for tissue regeneration.

### SUMMARY

In some embodiments, a cell suspension having therapeutic potential may comprise a population of cells derived from freshly disaggregated tissue, comprising at least 550,000 total cells per milliliter, wherein the population of cells has at least 30% viability, wherein at least 300,000 cells are viable, and wherein the median live cell diameter of the viable cells is greater than or equal to 9 micrometers; and in an effective amount of buffer for delivery. In some embodiments, the freshly disaggregated tissue may comprise epidermal tissue. In some embodiments, the population of cells may comprise keratinocytes, melanocytes, and fibroblasts. In some embodiments, the population of cells may be obtained by a process comprising obtaining a split-thickness skin sample, contacting the skin sample with an effective amount of warmed enzyme solution, contacting the skin sample with an effective amount of buffer, mechanically disaggregating the epidermal sample, contacting the skin sample with a second effective amount of buffer to create a buffered disaggregated tissue solution, and passing the buffered disaggregated tissue solution through a strainer, wherein the screen has a pore size of ≤100 micrometers.

A method for identifying a cell suspension with therapeutic potential for skin regeneration may comprise the steps of receiving a cell suspension, which may be prepared from a skin sample and comprises a population of viable cells and non-viable cells; measuring a value indicative of at least one characteristic of the cell suspension, wherein the at least one characteristic relates to one or more of cell count, cell viability, and cell size; and identifying the cell suspension as having therapeutic potential for skin regeneration based on a comparison between the value indicative of the at least one characteristic of the cell suspension and a predetermined threshold. In some embodiments, the at least one characteristic relating to cell count may comprise a quantity of viable cells and non-viable cells per unit volume, a quantity of viable cells per unit volume, a proportion of viable cells within the population of viable cells and non-viable cells, or a representative diameter of viable cells in the population of viable cells. In some embodiments, the predetermined threshold may comprise at least about 550,000 viable cells/ml, at least about 300,000 viable cells/ml, at least about 30% viability, or at least about 9 um live cell diameter. In some variations, the representative live cell diameter may comprise a mean diameter. In some variations, the cell suspension may comprise cells from the epidermal and dermal components from the skin sample suspended in a solution. In some variations, the cell suspension may comprise one or more additives to enhance therapeutic potential of the cell suspension. In some variations, the measuring the value indicative of the at least one characteristic of the cell suspension may be performed with a cell detection, measurement, and/or counting device, or may comprise the steps of staining the population of viable cells and non-viable cells with a viability reagent; and analyzing the stained viable cells and non-viable cells with the cell detection, measurement, and/or counting device.

A system for identifying a cell suspension with therapeutic potential for skin regeneration may comprise: a cell detection, measurement, and/or counting device configured to receive a cell suspension, wherein the cell suspension is prepared from a skin sample and comprises a population of viable cells and non-viable cells; and a processor coupled to the cell detection, measurement, and/or counting device, the processor configured to (i) measure a value indicative of at least one characteristic of the cell suspension received by the cell detection, measurement, and/or counting device, wherein the characteristic relates to one or more of cell count, cell viability, and cell size; and (ii) identify the cell suspension as having therapeutic potential for skin regeneration based on a comparison between the value indicative of the at least one characteristic of the cell suspension and a predetermined threshold. In some embodiments, the at least one characteristic relating to cell count may comprise a quantity of viable cells and non-viable cells per unit volume, a quantity of viable cells per unit volume, a proportion of viable cells within the population of viable cells and non-viable cells, and/or a representative diameter of viable cells in the population of viable cells. In some variations, the predetermined threshold may comprise at least about 550,000 cells/ml, at least about 300,000 cells/ml, and/or at least about 30%. In some embodiments, the predetermined threshold of the minimum diameter of viable cells in the population of viable cells may comprise about 9 um. In some embodiments, the representative diameter may comprise a mean diameter. In some embodiments, the cell suspension of the system may comprise dermal and epidermal cells from the skin sample suspended in a solution, one or more additives to enhance therapeutic potential of the cell suspension, or both. In some embodiments, the system measurement of the value indicative of the at least one characteristic of the cell suspension may comprise staining the population of viable cells and non-viable cells with a viability reagent; and analyzing the stained viable cells and non-viable cells with the cell detection, measurement, and/or counting device.

In some embodiments, a method of treating a subject with a cell suspension with therapeutic potential for skin regeneration may comprise preparing a cell suspension from a skin sample, wherein the cell suspension comprises a population of viable cells and non-viable cells; measuring a value indicative of at least one characteristic of the cell suspension, wherein the at least one characteristic relates to one or more of cell count, cell viability, and cell size; identifying the cell suspension as having therapeutic potential for skin regeneration for the subject based at least in part on a comparison between the value indicative of the at least one characteristic of the cell suspension and a predetermined threshold; and administering to a recipient region of the subject an amount of the cell suspension having therapeutic potential for skin regeneration. In some variations, the at least one characteristic relating to cell count may comprise a quantity of viable cells and non-viable cells per unit volume, a quantity of viable cells per unit volume, a proportion of viable cells within the population of viable cells and non-viable cells, or a representative diameter of viable cells in the population of viable cells. In some embodiments, the predetermined threshold may be at least about 550,000 viable cells/ml, at least about 300,000 viable cells/ml, at least about 30% viability, or at least about 9 μm live cell diameter. In some variations, the representative live cell diameter is a mean diameter. In some variations, the step of preparing the cell suspension is performed peri-operatively. In some embodiments, a time period between identifying the cell suspension as having therapeutic potential for skin regeneration and administering to the recipient region of the subject the amount of the cell suspension having therapeutic potential for skin regeneration may comprise less than about 5 hours. In some embodiments, the method may further comprise the steps of harvesting the skin sample from the subject and/or preparing the cell suspension from the skin sample comprises subjecting the skin sample to an enzyme to dissociate the skin sample. In some embodiments, the step of measuring the value indicative of the at least one characteristic of the cell suspension may be performed with a cell detection, measurement, and/or counting device. In some variations, the step of measuring the value indicative of the at least one characteristic of the cell suspension may comprise staining the population of viable cells and non-viable cells with a viability reagent; and analyzing the stained viable cells and non-viable cells with the cell detection, measurement, and/or counting device. In some embodiments, the step of administering the cell suspension to the subject may comprise spraying the cell suspension as airborne droplets on the recipient region of the subject, dripping the cell suspension on the recipient region of the subject, or any other technique of administration disclosed herein. In some embodiments, the recipient region of the subject may comprise skin with a wound, scar, or both, or skin with a pigmentation condition, or both. In some variations, the pigmentation condition is at least one selected from the group consisting of leukoderma, vitiligo, hyperpigmented scar, hypopigmented scar, and piebaldism.

In some variations, the cell suspension of one or more methods disclosed herein may comprise dermal cells and epidermal cells from the skin sample suspended in a solution, epidermal stem cells, activated keratinocytes, proliferating keratinocytes, basal keratinocytes, suprabasal keratinocytes, Merkel cells, Meissner's corpuscles, melanocytes, fibroblasts, Langerhans cells, endothelial cells, immune cells, and mesenchymal stem cells, one or more additives to enhance therapeutic potential of the cell suspension, or any combination thereof In some embodiments, the enzyme may comprise trypsin, recombinant trypsin, or trypsin-EDTA, dispase, collagenase, thermolysin, pronase, hyaluronidase, pancreatin, elastase, accutase, or papain. In some embodiments, the skin sample may provide an expansion ratio of between 1:1 and 1:200, between 1:1 and 1:80, between 1:4 and 1:85, or any range disclosed herein, with 1:80 being a preferred ratio. Depending on the ratio, the various measured quantities such as cells, viability, and phenotypical distribution may change as a result.

### BRIEF DESCRIPTION OF THE DRAWINGS

FIG. 1 is a flowchart illustrating an exemplary variation of a method for identifying a cell suspension with therapeutic potential for skin regeneration.

FIG. 2 is a flowchart illustrating an exemplary variation of a method of treating a subject with a cell suspension with therapeutic potential for skin regeneration.

FIG. 3 is a schematic diagram of an exemplary variation of a system for identifying cell suspension with therapeutic potential for skin regeneration.

FIG. 4A illustrates a box plot of total number of cells/ml in exemplary cell suspensions with high therapeutic potential.

FIG. 4B illustrates a box plot of cell viability in exemplary cell suspensions with high therapeutic potential.

FIG. 4C illustrates a box plot of total number of viable (live) cells/ml in exemplary cell suspensions with high therapeutic potential.

FIG. 4D illustrates a box plot of average viable (live) cell diameter in exemplary cell suspensions with high therapeutic potential.

FIG. 5 illustrates an exemplary gating strategy for evaluating cell suspensions.

### DETAILED DESCRIPTION

Methods and systems are disclosed for identifying a cell suspension with therapeutic potential for tissue regeneration. For example, a method and system for validating the use of a cell suspension for administration to a patient is described herein.

Individuals (e.g., patients, subjects, etc.) who experience trauma to their skin or experience other skin conditions may benefit from therapeutic intervention that promotes skin regeneration. For example, skin that has undergone trauma, such as wound, burn, etc., may not regenerate during the normal healing process, or may benefit from treatment to expedite or otherwise enhance healing. Skin with pigmentation disorders such as leukoderma, vitiligo, piebaldism, etc. may also benefit from treatment that can promote skin regeneration. Similarly, genodermatoses and aging may also benefit from this treatment.

In some variations, therapeutic interventions to promote healthy skin regeneration may include preparing a mixed cell suspension from a skin sample. The cell suspension may comprise living cells, non-living cells, and non-cellular components having therapeutic potential for skin regeneration. The prepared cell suspension may be applied to a region of skin in order to promote skin regeneration. However, the therapeutic potential of a cell suspension may vary. For instance, cell count, cell health (e.g., cell viability), and cell size, which impact the therapeutic potential of a cell suspension may vary. Furthermore, the therapeutic potential of a cell suspension may change depending on the methodology used to prepare the cell suspension. For instance, cellular extraction methods, amount, volumes, and/or type of any additives, and/or cellular manipulation methods employed to prepare the cell suspension may impact the therapeutic potential of the cell suspension.

In some embodiments, therapeutic interventions to promote healthy skin regeneration may include preparing a bioactive suspension derived from freshly disaggregated skin tissue. In such embodiments, one or more methods of producing a cell suspension disclosed herein may comprise a further step wherein all cells are removed from the cell suspension, prior to administration to the patient. In such an embodiment, the present invention may comprise counting the extracted cells and, in some embodiments, inferring from such count as to the composition of the remaining cell-free supernate. It is anticipated that higher counts of extracted cells may, in some embodiments, correlate with a supernate having higher therapeutic value than a supernate derived from a cell suspension containing lower cell counts.

A system and method for identifying therapeutic potential of a cell suspension is disclosed herein. In some variations, the method may include receiving a cell suspension prepared from a skin sample. The cell suspension may include viable cells and non-viable cells. The cell suspension may be analyzed to measure at least one value that may be indicative of at least one characteristic of the cell suspension. For example, a sample of the cell suspension may be analyzed to measure one or more characteristics comprising total cell count, total cell viability, cell viability percentage, and median live cell diameter. In some variations, a cell detection, measurement, and/or counting device with a customized gate (as further explained herein) may be used to measure the value indicative of the at least one characteristic (e.g., cell counts, cell viability, or the cell size) of the cell suspension. These measured values may be analyzed in order to identify or otherwise characterize therapeutic potential of the cell suspension. For example, in some variations each of one or more measured values may be compared to a predetermined threshold in order to identify the therapeutic potential of the cell suspension.

In some variations, the method and system disclosed herein may utilize at least one predetermined threshold for indicating the therapeutic potential of a cell suspension. For example, one or more threshold values associated with total cell count, total cell viability, cell viability percentage, and cell size (median live cell diameter) may be representative of the therapeutic potential of a cell suspension. A comparison of the measured values to the determined threshold values may signify whether or not a particular cell suspension is viable (e.g., having sufficient therapeutic potential). In addition to determining the therapeutic potential of a cell suspension, the technology disclosed herein may also assess cell phenotype and presence of different cellular populations.

The system and methodology disclosed herein may, in some variations, have application during point-of-care of an individual in need of a treatment for a skin condition, such as damaged skin, pigmentation disorders, genetic conditions, or for rejuvenation. For example, the technology disclosed herein may be used during point-of-care, such as in the operating room, emergency room, clinic, hospital, physician's office, dermatologist office, or bedside to the patient. In some variations, the technology disclosed herein may be performed perioperatively.

Additionally or alternatively, the system and method disclosed herein may be used to evaluate new techniques and procedures for preparing and administering cell suspensions with therapeutic potential for skin regeneration. For instance, if a new technique is defined and/or established for preparing and administering cell suspensions, the technology described herein may determine whether the new technique may result in cell suspensions with therapeutic potential. For example, the technology described herein may evaluate new techniques of cellular extraction, new techniques of cellular manipulation, techniques with new amounts of additives, etc., in order to determine if a resulting cell suspension may have therapeutic potential. Accordingly, the technology disclosed herein may be able to validate and verify newly established techniques for skin regeneration.

Exemplary Method for Identifying a Cell Suspension with Therapeutic Potential

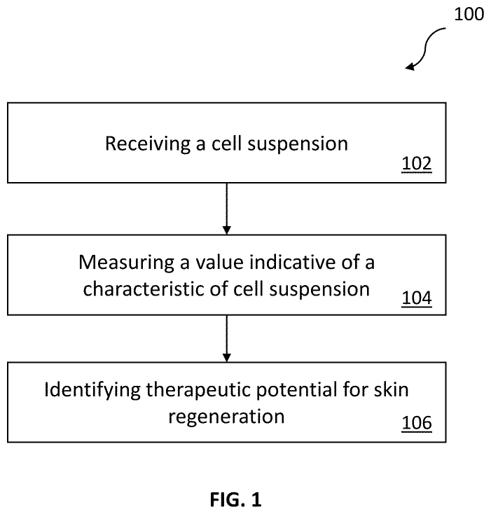

FIG. 1 is a flowchart illustrating an exemplary variation of a method 100 for identifying a cell suspension with therapeutic potential for skin regeneration. At 102, the method 100 may include receiving a cell suspension. At 104, a value that may be indicative of a characteristic of the cell suspension may be measured. At 106, the cell suspension may be identified as having therapeutic potential for skin regeneration.

### Receiving a Cell Suspension

A cell suspension may be prepared from a skin sample that may be harvested from a patient (e.g., an individual receiving the cell suspension as a therapeutic intervention) or from a donor (e.g., an individual not receiving the cell suspension). The prepared cell suspension may be received (e.g., at 102 in FIG. 1) by a user evaluating the therapeutic potential of the cell suspension. For example, the cell suspension may be received by a clinician, a dermatologist, a nurse, or a lab technician evaluating its therapeutic potential.

In some variations, the cell suspension may be prepared from a skin sample harvested from a patient that is intended to receive the cell suspension (e.g., an autologous skin sample). For example, the skin sample may be harvested from a healthy or undamaged skin region of a patient. In some variations, the skin sample may be harvested from a donor not intended to receive the cell suspension. For instance, the skin sample may be harvested from a donor that has skin suitable for a patient receiving the cell suspension.

In some variations, the skin sample may be developed in vitro. For instance, one or more in vitro culture methods may be implemented to grow a standardized skin cell culture. In some variations, the skin sample may be generated using a 3D printer. In some variations, the skin sample may be generated using one or more suitable methods from stem cells (e.g., pluripotent) or progenitor cells.

In general, the harvested skin sample may be thin. In some variations, the thickness of the harvested skin sample may vary with the region of the body from which the skin is harvested and/or the age of the patient. For example, the thickness of the harvested skin sample may be a minimum of 0.006 inches thick. In some embodiments, the harvested skin sample may be 0.001 to 0.006 inches thick. In some variations, the harvested skin sample may be 0.006 to 0.01 inches thick. In some preferred embodiments, the harvested skin sample may be 0.006 to 0.008 inches thick. In some variations, the harvested skin sample may be 0.01 to 0.10 inches thick. In some embodiments, the harvested skin sample may be greater than 0.10 inches thick.

In some variations, the skin sample may be harvested using a dermatome, scalpel, or other suitable instrument. In some variations, the harvested skin sample may comprise epidermal and dermal cells. The harvested skin cells may include, for example, keratinocytes inclusive of epidermal stem cells, proliferating keratinocytes (progenitor cells), basal keratinocytes, suprabasal keratinocytes, and activated keratinocytes. Additionally, the harvested skin may include mesenchymal stem cells, immune cells, fibroblasts, endothelial cells, Langerhans cells, Merkel cells, Meissner' s corpuscles, and melanocytes.

In some variations, the cell suspension may be prepared from the harvested skin sample, and the cell suspension may be configured to treat a region that has a treatment surface area greater than the donor surface area from which tissue for the cell suspension is harvested. For instance, in some variations, the cell suspension may be configured to have an expansion ratio of up to 1:80, meaning that 1 cm² of the harvested skin sample may create a cell suspension for treatment of an area of up to 80 cm². In some variations, the harvested skin sample may provide an expansion ratio between 1:5 and 1:80. In other embodiments, the harvested skin sample may provide an expansion ratio between 1:1 and 1:100. In other variations, the harvested skin sample may provide an expansion ratio between 1:1 and 1:200. In other embodiments, the harvested skin sample may provide an expansion ratio between 1:1 and 1:>200.

Generally, a cell suspension may be prepared by dissociating the harvested skin sample through physical and/or chemical disruption to obtain disaggregated cells from the skin sample. Physical disruption may include, for example, scraping the skin sample with a scalpel, mincing the tissue in the skin sample, physically cutting the layers apart, perfusing the skin sample, and/or the like. Chemical disruption may include digestion with one or more enzymes such as trypsin, dispase, collagenase, trypsin-EDTA, thermolysin, pronase, hyaluronidase, elastase, papain and pancreatin. Once the disaggregated cells are obtained, the method may include suspending the cells in a buffer or other nutrient solution to form a cell suspension, and in some variations, the cell suspension may be further processed, such as by straining to remove larger tissue aggregates, combining the cell suspension with one or more exogenous agents or other additives, etc. In some variations, the cell suspension may be prepared at least partially through manual methods, such any of the methods described in U.S. Pat. Nos. 9,029,140, 10,626,358, and/or U.S. patent application Ser. No. 16/935,977, each of which is incorporated herein its entirety by this reference. However, in some variations the cell suspension may be prepared at least partially through one or more automated methods, such as with a system similar to that described in U.S. Pat. No. 10,626,358 including a pestle or other suitable actual able tissue disaggregating member with a surface suitable for performing physical tissue disruption (e.g., grinding, scraping, shaving, etc.).

In some variations, preparing the cell suspension may include preparing an enzyme solution for use in chemically dissociating the cellular stratum in the skin sample. For example, an enzyme solution may be formed by mixing lyophilized enzyme with an appropriate volume of fluid (e.g., water). As discussed above, some non-limiting examples of the enzyme include trypsin, dispase, collagenase, trypsin-EDTA, thermolysin, pronase, hyaluronidase, elastase, papain, accutase, recombinant trypsin, and pancreatin. In some variations, the enzyme solution may have an amount of enzyme between about 0.05% and about 5% per volume of solution, between about 0.1% and about 5% per volume of solution, between about 0.25% and about 2.5% per volume of solution, or about 0.5% enzyme per volume of solution. In some embodiments, the amount of enzyme may be 5% to 10% per volume of solution. In some variations, the amount of enzyme may exceed 10% per volume of solution. In some variations, the enzyme solution may be heated to a target temperature (e.g., about 20° C., between about 30° C. and about 37° C., between about 33° C. and about 37° C., or about 37° C.). In some variations, the enzyme solution may be heated to a target temperature that is less than 20° C. In some embodiments, the enzyme solution may be heated to a target temperature that exceeds 37° C.

For instance, the enzyme solution may be heated by placing the enzyme solution into a heating well, or by using any suitable heating mechanism. In some variations, the enzyme solution may be heated for a suitable period of time until the target temperature is reached. For example, the enzyme mixture may reach its target temperature in under three minutes of being subjected to heating. Once heated to the suitable target temperature, the warmed enzyme solution may be suitable for use in processing a harvested tissue sample.

In some embodiments, the enzyme solution may be heated by means of an external warmer. In such embodiments, the external warmer may comprise a pouch having an electronic heating element. In other embodiments, the external warmer may comprise a blister pack assembly wherein a user may burst the blister pack to activate a chemical reaction that gives off sufficient heat to warm the enzyme. In other embodiments, the heating element may comprise a flame or other combustion element. In other embodiments, the heating element may comprise a radiator assembly that conducts heat indirectly to the enzyme solution.

In some variations, a kit similar to that described in U.S. Pat. No. 9,029,140 and/or U.S. patent application Ser. No. 16/935,977 (each of which was incorporated by reference above) may provide resources to prepare a suitable enzyme solution. For example, a kit may provide an enzyme vial of lyophilized enzyme, a vial of sterile water, and/or appropriate measurement or manipulation instruments. A diaphragm of the enzyme vial may be optionally wiped with sterile alcohol wipe and allowed to dry. A syringe may be inserted into an interior of the water vial and an appropriate volume of water may be drawn from the water vial into the syringe. The volume of water may then be injected from the syringe into the interior of the enzyme vial and mixed gently (e.g., without shaking to avoid foaming) until the enzyme is dissolved in the water to form an enzyme solution. The enzyme solution may be drawn back into the syringe for distribution into a heating well or other suitable heating mechanism.

As described above, a method of preparing a cell suspension may include chemically dissociating the harvest skin tissue sample, such as with the prepared enzyme solution. For example, the harvested skin sample may be submerged in the heated enzyme solution (e.g., by submerging the skin sample in a heating well into which the enzyme solution was dispensed) such that the enzyme solution may break down protein-protein interactions. For example, the skin sample may be submerged in the enzyme solution for a suitable incubation period, such as between about fifteen to about twenty minutes. Thicker skin samples may be submerged in the enzyme solution for a longer period of time (e.g., up to sixty minutes). In some variations, thicker skin samples may be submerged in an enzyme solution with a higher concentration of the enzyme.

In some variations, sufficient incubation in the enzyme solution may be determined with a test scrape for cell disaggregation, such as by gently scraping an epidermis edge of the sample with a scalpel to test whether epidermal cells are easily removed. For example, the skin sample may be removed from the heated enzyme solution and placed dermal side down on an appropriate surface, such as with sterile forceps or scalpel. The epidermis edge of the skin sample may be scraped gently with the scalpel to test if the cells disaggregate (e.g., if the epidermal cells separate easily). If the cells disaggregate, the scraping may be stopped. If the cells do not disaggregate, the skin sample may be returned to the heated enzyme solution for a period of time (e.g., about five to ten minutes), and then removed for additional test scraping to determine whether the cells disaggregate.

If the cells scrape freely, the skin sample may be placed submerged in a buffer solution to neutralize and/or rinse off any residual enzyme solution on the skin sample. In some variations, the buffer solution may include at least a serum. In such situations, the buffer solution may be used to rinse off any residual enzyme solution on the skin sample or inactivate residual enzyme solution on the skin sample. In some variations, the buffer may have the characteristics of being (i) free of at least xenogenic serum, (ii) capable of maintaining the viability of the cells until applied to a patient, and (iii) suitable for direct application to a region on a patient. The buffer may be anything from a basic salt solution to a more complex solution. In some variations, the buffer may be free of all serum but may contain various salts that resemble the substances found in body fluids (e.g., physiological saline). Phosphate or other non-toxic substances may also be used as buffer in order to maintain the pH at approximately physiological levels. A suitable buffer solution may include, for example, Hartmann's solution.

As used herein, an effective amount of buffer is an amount sufficient to accomplish the task at hand, such as but not limited to the amount of buffer required to rinse off residual enzyme, suspend cells in suspension, or any other element described herein. Examples of an effective amount range from 1 ml to 10 mls, 10 mls-100 mls, and 100 mls to 1000 mls, with any amount therein having been contemplated for use with the present disclosure and invention.

After application of the buffer, the cellular stratum of the skin sample may be separated through mechanical disaggregation. For example, epidermal cells may be scraped from the skin sample using an instrument such as a scalpel, and then continued scraping may reduce the dermis until the dermis has disintegrated or nearly disintegrated. The disaggregated cells may then be further rinsed and suspended in a suitable amount of buffer or other nutrient solution. Among the resulting disaggregated cells may include cells capable of proliferation (including but are not limited to keratinocytes inclusive of epidermal stem cells, proliferating keratinocytes, basal keratinocytes, suprabasal keratinocytes, and activated keratinocytes, Langerhans cells, Merkel cells, Meissner's corpuscles, fibroblasts and melanocytes, etc.). Furthermore, the cell suspension may include a population of viable cells (e.g., living cells) and non-viable cells (e.g., non-living cells).

To avoid excessively large cellular aggregates or tissue debris in the cell suspension the suspension may also be strained. Any suitable cell strainer capable of separating excessively large tissue aggregates from the suspension may be used to strain the cell suspension. In some variations, the cell strainer pore size is between 50 μm and 200 μm, or between 75 μm and 150 μm, with 100 μm being one specific example. After straining, the cell suspension may be diluted to produce an appropriate cell density suitable for the purpose to which the suspension is to be used.

In some embodiment, the step of diluting the cell suspension may comprise adding buffer to the cell suspension. In other embodiments, the step of diluting the cell suspension may comprise adding hyaluronic acid to the cell suspension. In some embodiments, the step of diluting the cell suspension may comprise diluting the cell suspension in any means known in the art.

In some variations, the cell suspension prepared as described above, may have sufficient homogeneity to enable the measurement of one or more characteristics of the cell suspension (e.g., at 104 in FIG. 1). Alternatively, the cell suspension prepared as described above may be gently mixed to obtain a homogenous suspension. If cell clumps are visible, the cell suspension may be mixed (e.g., 1000 μl pipette) to get an even cell suspension.

In some variations, the cell suspension may include one or more additives that may enhance the therapeutic potential of the cell suspension. For example, exogenous agent(s) or component(s) such as heat shock protein(s), hyaluronic acid, platelet-enriched plasma, growth factor(s), cytokine(s), and/or adipose stem cells can be supplied to the population of cells in the cell suspension. Useful growth factors and cytokines include but are not limited to, epidermal growth factor, transforming growth factor-α and -β, hepatocyte growth factor, vascular endothelial growth factor, platelet derived growth factor, fibroblast growth factor 1 and 2, insulin-like growth factor 1 and 2, interleukin 8, connective tissue growth factor, and keratinocyte growth factor. Adipose stem cells include various stem cells having proliferative and differentiating potentials collected from adipose tissues. Other examples of additives and processes for enhancing a cell suspension with such additives are described in U.S. Patent Publication No. US 2015/0079153, which is incorporated herein by reference in its entirety.

### Measuring a Value Indicative of a Characteristic of the Cell Suspension

A user may transfer a sample of the prepared cell suspension (e.g., cell suspension received at 102 in FIG. 1) into a cell detection, measurement, and/or counting device (e.g., Moxi Go II™) to measure one or more values indicative of a characteristic of the cell suspension (e.g., at 104 in FIG. 1). In some variations, the one or more values may be measured immediately after the preparation of the cell suspension. Alternatively, the time period between preparing the cell suspension and measuring one or more values may be within the range of between about 1 minute and about 2 hours. In such variations, the cell suspension may be prepared bedside and administered to the patient within a few hours. Alternatively, the time period between preparing the cell suspension and measuring at least one value indicative of at least one characteristic of the cell suspension may range from a few days to a few months to a few years. In such variations, the cell suspension may be prepared at a location away from the patient and may be transported (e.g., shipped) so as to be administered to the patient. In some variations, the cell detection, measurement, and/or counting device may detect the soluble proteins in the cell suspension.

Additionally, in preferred embodiments, the step of measuring a value indicative of a characteristic of a cell suspension 104 may comprise measuring all of the following characteristics: total cells per milliliter, percentage viability, total viable cell count, and median live cell diameter of the viable cells. In some embodiments, the step of measuring a value indicative of a characteristic of a cell suspension 104 may comprise measuring three or more of the following characteristics: total cells per milliliter, percentage viability, total viable cell count, and median live cell diameter of the viable cells. In some embodiments, the step of measuring a value indicative of a characteristic of a cell suspension 104 may comprise measuring three two or more of the following characteristics: total cells per milliliter, percentage viability, total viable cell count, and median live cell diameter of the viable cells. In one example method for measuring one or more values of a cell suspension 104, a small aliquot (e.g., minimum of 30 μl, recommended value of 100 μl) of homogeneously mixed cell suspension may be transferred to a vessel (e.g., Eppendorf tube). The cell suspension may be mixed with a reagent (e.g., 15 μl of cell suspension may be mixed with 135 μl of the reagent to create a 1:10 dilution) to create a dilution. The vessel may be incubated protected from light at room temperature for an appropriate amount of time (e.g., 5 minutes). For example, the incubation may occur in a dark room, in an opaque container or under an opaque cover, and/or the like. The cell suspension may then be transferred to a suitable analyzer (e.g., a flow cytometer) by pipetting the cell suspension up and down to evenly mix the suspension and then applied to the analyzer. The analyzer may measure values associated with one or more characteristics of the cell suspension.

Some non-limiting examples of characteristics may include cell count, cell viability, cell size, a combination thereof, and/or the like.

### Total Cell Count

As discussed above, the cell suspension may include both viable cells and non-viable cells. In some variations, the characteristic of total cell count may comprise a quantity of total number of cells (e.g., both viable cells and non-viable cells) in per unit volume of the cell suspension. For example, the cell count may indicate the concentration of viable cells and non-viable cells per unit volume of the cell suspension. In some variations, the cell count may indicate a quantity of just the viable cells per unit volume of the cell suspension. For example, the cell count may indicate the concentration of just the viable cells per unit volume of the cell suspension.

In some embodiments, a cell suspension having therapeutic potential per the present disclosure may comprise a value of at least at least 550,000 total cells per milliliter.

### Total Viable Cell Count

In some variations, the characteristic of total viable cell count may comprise the total number of viable cells per unit volume.

In some embodiments, a cell suspension having therapeutic potential per the present disclosure may comprise a population of cells wherein a value of at least 300,000 cells per milliliter are viable.

### Cell Viability Percentage

In some variations, the characteristic of cell viability percentage may comprise a proportion of viable cells within a population of both viable cells and non-viable cells (e.g., total cell count). For example, cell viability percentage may be a percentage of quantity of viable cells per unit volume relative to total number of cells per unit volume.

In some embodiments, a cell suspension having therapeutic potential per the present disclosure may comprise a population of cells comprising a value of at least 30% viable cells.

### Median Live Cell Size (Diameter)

In some variations, the characteristic of median live cell diameter (or size herein) may comprise a mean diameter of viable cells in the cell suspension. Alternatively, the cell size may be a median diameter of viable cells in the cell suspension. In yet another alternative variation, the cell size may be a mode diameter of viable cells in the cell suspension.

In some embodiments, a cell suspension having therapeutic potential per the present disclosure may comprise a population of cells wherein the median live cell diameter is greater than or equal to 9 micrometers.

### Measuring the Values Related to Characteristics of the Cell Suspension

One or more values relating to one or more characteristics of the cell suspension may be measured using a cell detection, measurement, and/or counting device. The cell suspension may be stained and incubated (e.g., as discussed above) for processing and measurement. For instance, a staining mechanism may differentiate between live cells and dead cells in the cell suspension. In some variations, the cell suspension may be stained using propidium iodide as a staining method. However, any suitable staining mechanism may be used in conjunction with the cell detection, measurement, and/or counting device in order to process and measure the characteristics of the cell suspension.

The stained cell suspension may be transferred to the cell detection, measurement, and/or counting device. The cell detection, measurement, and/or counting device may include one or more components (e.g., a fluidics systems) to streamline the flow of cells into a single-file. As the cells enter the cell detection, measurement, and/or counting device in a single-file, the cells may interact with an optical system included in the cell detection, measurement, and/or counting device. For instance, light (e.g., laser light) from the optical system may intercept the cells. Based on the physical features of the cells, some of the intercepted light may be scattered. This scattered light may be correlated with characteristics of the cell suspension.

The measurement of values and hence characteristics may be built upon the principle of gating. For instance, gates and regions may be placed around a population of cells. Forward scatter gating may identify cells based on cell size while side scatter gating may identify cells based on their granularity. Backgating may identify the cells in the cell suspension to confirm staining pattern (e.g., an absence and/or presence of stain) and/or gating strategy. The correlation of the scattered light with the one or more characteristics of the cell suspension using a gating strategy may be digitized via electronics included in the cell detection, measurement, and/or counting device. In this manner, the cell detection, measurement, and/or counting device may measure characteristics of the cell suspension.

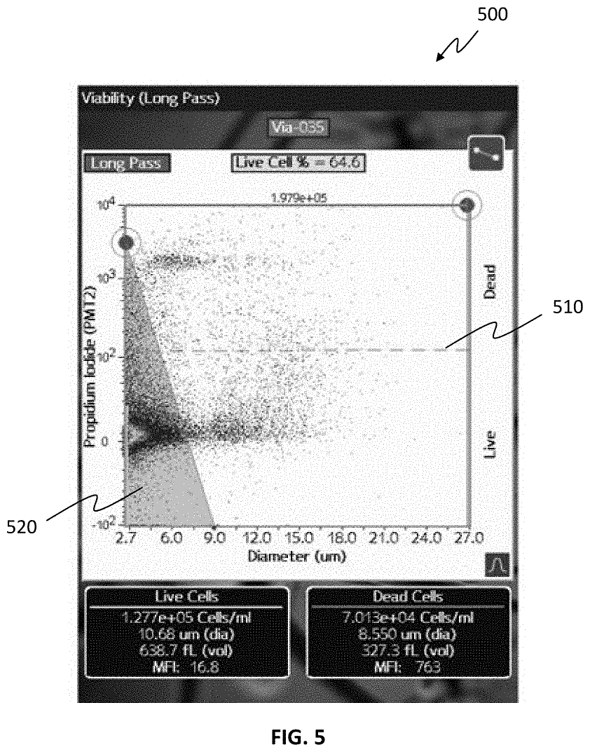

In some variations, a gating strategy may, for example, distinguish between live and dead cells (e.g., via a staining mechanism) and/or exclude particles considered as debris (e.g., from live or dead cells) from the analysis when characterizing a cell suspension. For example, FIG. 5 depicts a plot 500 illustrating an example gating strategy for characterizing a cell suspension. After staining particles within the cell suspension (e.g., with propidium iodide or other suitable staining agent) the cell suspension may be processed by a cell detection, measurement, and/or counting device as described above. Any particles having a fluorescence above a live/dead cutoff threshold 510 of about 1.1×10² may be considered “dead,” while any particles having a fluorescence below this live/dead cutoff threshold may be considered “live.” Furthermore, the region 520 represents thresholding used to distinguish between debris and cells. For example, particles having a diameter of less than about 9 μm (and having a diameter and fluorescence that falls within the region 520) may be considered debris and excluded from further analysis. Particles having a diameter and fluorescence that falls outside of the region 520 may be considered cells that contribute to total cell number in the cell suspension. Within this total cell population having characteristics that fall outside of the region 520, particles that are above the live/dead cutoff threshold 510 may be considered dead cells, while particles that are below the live/dead cutoff threshold 510 may be considered live cells, for purposes of identifying therapeutic potential of the cell suspension as described in further detail below.

### Identifying Therapeutic Potential For Skin Regeneration of a Cell Suspension

In some variations, in order to identify the therapeutic potential of cell suspension, the measured characteristics may be compared to one or more predetermined thresholds. More specifically, one or more threshold values representing the therapeutic potential of the cell suspension may be associated with the characteristics. The measured values may be compared with the threshold values. The therapeutic potential of the cell suspension may be identified based on this comparison.

In some variations, one or more threshold values may be associated with each characteristic of the cell suspension. For instance, a minimum threshold value may be associated with each characteristic of the cell suspension. Alternatively, a maximum threshold value may be associated with each characteristic of the cell suspension. In yet another alternative variation, a minimum threshold value and a maximum threshold value may be associated with each characteristic of the cell suspension.

In some variations, if the measured value for each specific characteristic meets the threshold criteria of that characteristic, then the cell suspension may be considered as having sufficient therapeutic potential. For example, if the measured value for each of cell size, cell viability, and cell count meet their respective threshold criteria, then the cell suspension may be considered having therapeutic potential. Alternatively, if the measured value of at least one characteristic meets the threshold criteria of that characteristic, then the cell suspension may be considered having therapeutic potential. For example, if the measured value of cell size meets the threshold criteria for cell size, then the cell suspension may be considered therapeutic. Similarly, if the measured value of cell viability meets the threshold criteria for cell viability, then the cell suspension may be considered viable.

In some variations, all the characteristics may be associated with collective threshold values. For instance, all the characteristics may be collectively represented as a polynomial. One or more threshold values (e.g., minimum value, maximum value, and/or range) may be collectively associated with that polynomial. If the measured characteristics collectively meet the threshold criteria, the cell suspension may be considered viable. In yet another variation, each individual characteristic may be associated with one or more respective threshold values. If at least two or more measured characteristics meet their respective threshold value, then the cell suspension may be considered viable. For example, if the measured cell count and the measured cell size meet their respective cell criteria, the cell suspension may be considered viable even if the measured cell viability may not meet its respective criteria.

In some variations, the identification of the cell suspension as having sufficient therapeutic potential (and/or the identification of threshold values for the measured characteristics not being met) may be communicated to a user. Additionally or alternatively, one or more of the measurements for measured characteristics may be communicated to a user. Such information may, for example, be communicated to a user through a user interface associated with the cell detection, measurement, and/or counting device. As another example, such information may be communicated through a user interface associated with another computing device (e.g., a computer, mobile computing device, etc.) that is in communication (e.g., through an application programming interface (API)) with the cell detection, measurement, and/or counting device and receives the measured characteristics of the cell suspension and/or results of comparing the measured characteristics with one or more predetermined thresholds. The identification of a cell suspension may, for example, be presented visually through a display device, and/or presented in an audible manner through an audio speaker device, in any suitable combination of text, color coding, symbols, sound effects, and/or the like.

### Exemplary Threshold Values

Table 1 below shows exemplary threshold values for cell count, cell viability, and cell size. In some variations, these thresholds may be used to evaluate therapeutic potential of a cell suspension having up to a 1:80 expansion ratio.

TABLE 1

| Characteristics | Minimum Value Requirement (Threshold) |
|---|---|
| Total Cell Count | >550,000 cells/ml |
| Cell Viability | >30% |
| Total Viable Cells | >300,000 cells/ml |
| Mean Live Cell Diameter | ≥9 μm |

Total Cell Count—In some variations, the threshold value for cell count (e.g., total cell count including viable cells and non-viable cells per unit volume) may be about 550,000 cells/ml or higher. For example, the minimum threshold value for cell count (e.g., total cell count including viable cells and non-viable cells per unit volume) may be 550,000 cells/ml. Accordingly, in some variations, if the measured total cell count value is lower than 550,000 cells/ml, the cell suspension may not be considered to possess therapeutic value. Viable Cell Count—In some variations, the threshold value for viable cell count (e.g., viable cell count per unit volume) may be about 300,000 cell/ml or higher. For example, the minimum threshold value for cell count (e.g., viable cell count per unit volume) may be 300,000 cells/ml. Accordingly, in some variations, if the measured viable cell count value is lower than 300,000 cells/ml, the cell suspension may not be considered to possess therapeutic value.

Cell Viability Percentage—In some variations, the threshold value for cell viability may be about 30% or higher. For example, the minimum threshold value for cell viability may be 30%. Accordingly, in some variations, if the measured cell viability is lower than 30%, the cell suspension may not be considered to possess therapeutic value.

In some embodiments, the cell count may be high enough such that within the population of cells, at least 300,000 cells are viable, but the proportion of viable cells do not surpass 30%. For example, in a total cell count of 2,000,000, were 350,000 cells to be viable, the percentage of viable cells would not surpass 30%, despite meeting the 300,000 viable cell requirement previously discussed, and hence would not be suitable for the present composition and method.

Median Live Cell Diameter—In some variations, the threshold value for live cell size may be an average diameter of about 9 μm or higher. For example, the minimum threshold value for the average live cell diameter may be 9 μm. Accordingly, in some variations, if the measured viable cell size is lower than 9 μm, the cell suspension may not be considered to possess therapeutic value.

Exemplary Method for Treating a Patient with a Cell Suspension Having Therapeutic Potential

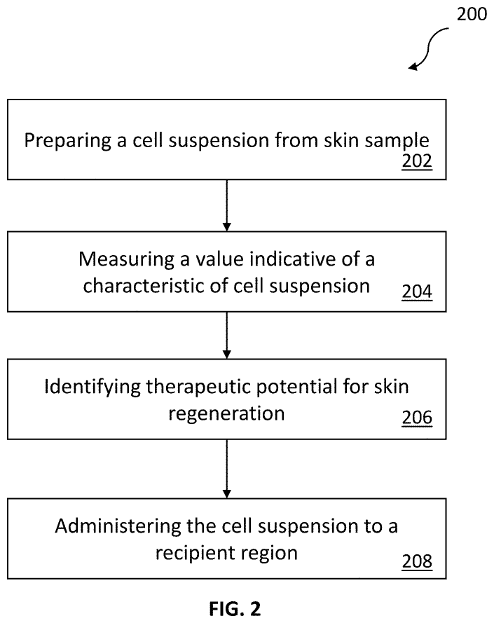

FIG. 2 is a flowchart illustrating an exemplary variation of a method 200 for treating a patient with a cell suspension with therapeutic potential. At 202, the method may include preparing a cell suspension from a skin sample. The method for preparing the cell suspension may be similar to the method described above. The cell suspension may be prepared by first harvesting the skin sample as described above. At 204, the method may include measuring at least one value that may be indicative of the cell suspension (e.g., similar to 104 in FIG. 1). In preferred embodiments, the step of measuring a value indicative of a characteristic of a cell suspension may comprise of measuring all of the following characteristics: total cells per milliliter, percentage viability, total viable cell count, and median live cell diameter of the viable cells. In some embodiments, the step of measuring a value indicative of a characteristic of a cell suspension 204 may comprise measuring three or more of the following characteristics: total cells per milliliter, percentage viability, total viable cell count, and median live cell diameter of the viable cells. In some embodiments, the step of measuring a value indicative of a characteristic of a cell suspension 204 may comprise measuring three two or more of the following characteristics: total cells per milliliter, percentage viability, total viable cell count, and median live cell diameter of the viable cells.

The cell suspension may be identified as having therapeutic potential for skin regeneration at 206 (e.g., similar to 106 in FIG. 1), such as based on comparisons between the measured values and predetermined thresholds of that value. At 208, the method may include administering the cell suspension having therapeutic potential. The cell suspension may be administered to a recipient region of a patient for skin regeneration.

In some variations, a time period between identifying the cell suspension as having therapeutic potential for skin regeneration (e.g., at 206) and administering to the recipient region the cell suspension (e.g., at 208) may be less than 2 hours, less than 1 hour, less than 30 minutes, or less than 15 minutes. In some variations, a time period between identifying the cell suspension as having therapeutic potential for skin regeneration (e.g., at 206) and administering to the recipient region the cell suspension (e.g., at 208) may be more than a few days, more than a few months, or more than a few years. In an alternate variation, a patient's skin may be harvested, or obtained as a result of a panniculectomy or other surgical procedure, stored in a tissue bank, and then processed a few days, few months, or a few years thereafter, at which time the assessment of the therapeutic potential of the cell suspension may be conducted using the aforementioned methods.

The method 200 may be used to treat a patient in need of therapeutic skin treatment (e.g., for treatment, healing, reconstructing, resurfacing, repigmentation and/or regeneration of epithelial tissues). For example, in some variations the patient may suffer from an acute wound (e.g., burn or laceration caused by trauma), an artificially created wound (e.g., in a graft donor site, aesthetic indication, plastic procedure or dermal treatment, etc.), a chronic wound (e.g., neuropathic ulcers, pressure sores, arterial and venous or mixed arterio-venous ulcers, diabetic ulcers, etc.). As another example, in variations the method may be used to treat indications such chronic wounds, scar revision, pigmentation issues, hypotrophic scars, vitiligo, piebaldism, leukoderma, skin aesthetic procedures and/or cosmetic rejuvenation procedures (e.g., treatments for wrinkles, acne scars, etc.).

In some variations, the cell suspension may be administered directly to a recipient region 208. For example, if the recipient region includes wound, burn, altered pigmentation, or other malady with dermal elements remaining or is partial-thickness, then the cell suspension may be applied directly to the recipient region. In some embodiments, the recipient region may have been previously excised, debrided, lasered, dermabraded, or otherwise prepared to receive a therapeutic cell suspension. In some embodiments, the cell suspension may be delivered via microneedling or intradermal injection.

The cell suspension may be applied using one or more suitable application techniques. In some variations, at least a portion of the cell suspension may be applied via spray application technique. For example, a spray applicator such as a syringe, a spray nozzle, a syringe attached to a spray nozzle, a powered spray device such as a “spray gun” or other such delivery devices may be used to apply the cell suspension. The spray applicator may be held such that the nozzle may face the wound. The spray applicator may be held at approximately 10 cm from the most elevated point of the recipient region such that the first drop of cell suspension falls onto the recipient region. The cell suspension may be sprayed from the most elevated point of the recipient region such the any run-offs may cover more dependent areas of the recipient region. A fine mist of cell suspension may be delivered to the recipient region. In some variations, if the recipient region is large, the spray applicator may be moved in continuous motion from one part of the recipient region to another part of the recipient region as the cell suspension is being sprayed.

In some variations (e.g., requiring an application of lesser than 2 ml of the cell suspension), at least a portion of the cell suspension may be applied via drip application technique. In such variations, a syringe may be held adjacent to or a few millimeters away from an elevated point of the recipient region such that the cell suspension is carefully dripped onto the recipient region. Any run-off may cover more dependent areas of the recipient region.

Moreover, in some embodiments, the cell suspension may be formulated as an injectable solution. In such embodiments, the cell suspension may comprise more or less water or other liquid solution so as to ensure that the viscosity of the bioactive solution is appropriate for administration by injection.

In another variation, the cell suspension may be prepared by combining certain tissue regeneration factors with cell culture media, Lactated Ringers solution, or other suitable media or solution.

In some embodiments, the cell suspension may be combined with one or more scaffolding elements prior to the application of this combination to a treatment site.

Alternatively, such as if the recipient region includes full-thickness wound, burn, altered pigmentation, or other malady with no continual dermal elements present, then the cell suspension may be applied in combination with a meshed split-thickness skin graft or another scaffolding material.

In some embodiments, the scaffolding material may comprise a skin graft, an extracellular matrix, or other matrix material. Examples of scaffolding elements comprising skin grafts include, but are not limited to, one or more of human skin, a meshed split-thickness skin graft, a split-thickness skin graft, a split-thickness human skin sample, a full thickness human skin sample, human skin epidermis, human skin dermis, human skin epidermal-dermal junction tissue, human mesenchymal stem cells, human adipose tissue, non-human epidermis, non-human dermis, non-human epidermal-dermal junction tissue, non-human mesenchymal stem cells, and non-human adipose tissue.

In various embodiments, the extracellular matrix or matrix material used can be synthetic or biological, such as collagen, alginate, alginate beads, agarose, fibrin, fibrin glue, fibrinogen, blood plasma fibrin beads, whole plasma or components thereof, laminins, fibronectins, proteoglycans, HSP, chitosan, heparin, and/or other synthetic polymer or polymer scaffolds and solid support materials, such as wound dressings, that could hold or adhere to cells. In some embodiments, the scaffolding material may comprise a tissue or organ regeneration system (in vivo or in vitro).

In some embodiments, the scaffolding element may comprise polymeric materials, such as but not limited to synthetic polymer films, foam dressings, hydrocolloids, alginate dressings, and hydrogels, polymeric foam, polymeric hydrogels, polymeric alginates, polymeric hydrocolloides, passive synthetic polymer dressings, interactive synthetic polymer dressings, polymer films, polyurethane (PU), semi-occlusive film, occlusive film, polyurethane foam film dressing, hydrophilic polymeric foam dressing, polysaccharides, agar, alginate, alginate dressings, carrageenans, pectin, gelatin, carboxymethylcellulose, crosslinked polymers (hydrophilic) such as polyvinylpyrrolidone, polyacrylamide, and polyethylene oxide, poly(lactide-co-glycolide) (PLGA), polyethylene glycol (PEG), PEG blended with chitosan and PLGA, polycaprolactone (PCL), electrospun PCL fibers, fucoidan, sulphated polysaccharides, silk sericin, acetobacter xylinum utilizes carbon from nutrition media and form beta 1-4 glucose in the form of linear chains, keratin, hyaluronic acid dressings, homoglycans such as starch, cellulose, and dextran, pallulan, yeast, grains and fungi yield beta glucans which can form double and triple helix resistant gel, bovine serum albumin, bi-layered bioengineered skin substitute (BBSS) acellular (collagen) matrixes from porcine small intestines, cultured allograft and autograft epidermal sheets, allografts from cadaveric skins, tissue-engineered skin substitutes, collagen-glucosamine skin scaffolds, sericin (silkworm) matrices, dermal substitutes, animal derived acellular xenografts, shark derived matrix of bovine collagenase chondroitin-6-sulfate and disposable silicone sheet, nylon mesh crosslinked with the porcine collagen, bi-layered substitute with a removable semi-permeable silicone layer, acellular dermal allografts, cadaveric-derived acellular dermal allografts, temporary bioengineered skin substitute, neonatal fibroblasts cultured on nylon fiber that are embedded into a silastic layer for 4-6 weeks and form dense cellular tissue, absorbable polyglactin scaffold colonized with allogenic neonatal fibroblasts, composite grafts, collagen scaffold, cultured fibroblasts and a layer of stratified cultured human keratinocytes, cultured neonatal keratinocytes and bovine collagen, composite skin graft, skin equivalent, organo-typical skin substitute, compositions of both living dermis and epidermis, composite bi-layer products, bi-layer bioengineered skin, fibroblast seeded scaffolds, adipose-derived human lipoaspirate from embryonic mesenchyme, non-contact radiant heat bandages, micro- and nanoparticulate systems, nanogels, novel nanofibrous chitosan-Fb scaffold, electrospun nanofibrous hybrid wound-dressing from PCL/collagen, electrospun nanofibers enriched with quercetin and ciprofloxacin hydrochloride, PCL/gelatin hybrid composite mats, microfibrous constructs with agAg nanoparticles, fibroblast seeded PU/SF scaffolds, biocompatible natural carbohydrate polymeric dressings including chitosan, microbe-derived polysaccharides such as microbial cellulose, and any other polymeric biomaterial known to be compatible with the present disclosure.

In some embodiments, the scaffolding element may comprise a fibrin glue, also known as a fibrin sealant or fibrin tissue adhesive, any other hemostatic, tissue sealant, and tissue adhesive; primary polymer-based dressing, or a primary wound spray dressing such as but not limited to wound spray, liquid bandage, first aid spray, antibacterial polymersome-based wound dressing spray; oil based bactericides/virucides spray dressings; and any other spray or liquid-applied bandage substitute.

In some embodiments, the scaffolding element may comprise bioprinted tissue, such as by way of illustration and not limitation, bioprinted skin tissue, a bioprinted gel configured for application to a skin wound. In some embodiments, the scaffolding may comprise a different type of bioprinted tissue, such as muscle, bone, nerve, or connective tissue.

In some embodiments, a cell suspension therapeutic may comprise a cell suspension made according to at least one embodiment disclosed herein in combination with at least one scaffolding element. By way of illustration and not limitation, such a cell suspension therapeutic may comprise, in some embodiments, a population of cells derived from freshly disaggregated tissue, comprising at least 550,000 total cells per milliliter, wherein the population of cells has at least 30% viability, wherein at least 300,000 cells are viable, and wherein the median live cell diameter of the viable cells is greater than or equal to 9 micrometers, an effective amount of buffer, and at least one scaffolding element.

Exemplary System for Identifying a Cell suspension with Therapeutic Potential

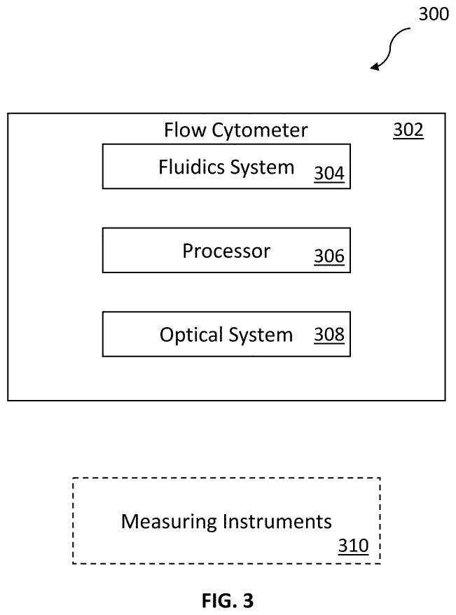

FIG. 3 is a schematic diagram of an exemplary variation of a system 300 for identifying cell suspension with therapeutic potential for skin regeneration. The system may include a cell detection, measurement, and/or counting device 302 to process and analyze characteristics of a cell suspension in order to identify therapeutic potential. The cell detection, measurement, and/or counting device 302 may include fluidics system 304, processor 306, and optical system 308. In some variations, the system 300 may optionally include measuring devices 310.

As used herein, the term “flow cytometer” 302 is to be construed as encompassing any cell detection, measurement, and/or counting device or any technique useful to accomplish the same purpose. Indeed, it is expressly contemplated that the cell detection, measurement, and/or counting device may optionally comprise but is not limited to a flow cytometer, a hemocytometer, fluorescent microscope, or other device known in the art useful to accomplish the same purpose. Additionally or alternatively, techniques for cell detection, measurement, and/or counting may also be used in place of cell detection, measurement, and/or counting device 302, such as but not limited to manual cell counting, LIVE/DEAD staining protocols, and fluorescent microscopy using any device known in the art to be useful to accomplish the same purpose. In some variations, the cell detection, measurement, and/or counting device 302 may be any suitable existing flow cytometer as defined herein such as Moxi Go II™, Guava® Muse® Cell Analyzer, Attune NxT Flow Cytometer™, BD Accuri™ C6 Plus Flow Cytometer, etc. The cell detection, measurement, and/or counting device 302 may include one or more gates, thereby implementing the principles of gating. In some variations, the cell detection, measurement, and/or counting device 302 may include fluidics system 304. The fluidic system 304 may include tubes, pumps, valves, etc. to streamline the flow of cells in the cell suspension. For instance, the tubes, pumps, and valves may be integrated and/or coupled in a way so as to form a hydrodynamic focusing region within the cell detection, measurement, and/or counting device. The cells are analyzed in the hydrodynamic focusing region of the cell detection, measurement, and/or counting device. In some variations, the cell detection, measurement, and/or counting device 302 may include optical system 308 to analyze the characteristics of the cell suspension. The optical system 308 may include one or more light sources. For instance, the optics system 308 may include a laser emitting laser beam(s). In some variations, the optical system 308 may also include one or more detectors to measure an amount of scattered light (e.g., light scattered following interception with cells in the cell suspension). The optical system 308 may include a forward scatter detector and a side scatter detector.

In some variations, the cell detection, measurement, and/or counting device 302 may include a processor 306 to analyze the detected scattered light and to digitize the measured scattered light. The processor 306 may be any suitable processing device configured to run and/or execute a set of instructions or code, and can include one or more data processors, image processors, graphics processing units, digital signal processors, and/or central processing units. The processor 306 may be, for example, a general purpose processor, a Field Programmable Gate Array (FPGA), an Application Specific Integrated Circuit (ASIC), and/or the like.

In some variations, the cell suspension may be stained before being transferred to the cell detection, measurement, and/or counting device 302 for analysis. For example, the cell suspension may be stained using propidium iodide as a staining method. However, any suitable staining mechanism may be used in conjunction with the cell detection, measurement, and/or counting device 302 in order to process and measure the characteristics of the cell suspension. The stained cell suspension may be transferred to the cell detection, measurement, and/or counting device 302. For example, the stained cell suspension may enter the cell detection, measurement, and/or counting device 302 via the fluidics system 304. The fluidics systems 304 may streamline the flow of cells into a single file. For instance, the hydrodynamic focusing region may enable streamlining the flow of cells. The laser included in the optical system 308 may intercept the flow of cells. Depending on the physical features of the cells, some of the intercepted light may be scattered. For example, light may scatter from the edges of the cells. The forward scatter detector included in the optical system 308 may implement forward scatter gating and may identify cells based on the cell size. Side scatter detector included in the optical system 308 may implement side scatter gating and may identify cells based on their granularity. The processor 306 may correlate the scattered light with one or more characteristics of the cell suspension. In this manner, the cell detection, measurement, and/or counting device 302 may measure characteristics of the cell suspension.

In some variations, the cell detection, measurement, and/or counting device 302 may be optionally coupled to a printing device to print the measured characteristics, other measurements related to the measured characteristics, and/or scatter plots and/or box plots of measured characteristics of the cell suspension. In some variations, the cell detection, measurement, and/or counting device 302 may be coupled to a display (e.g., display screen of a laptop, desktop, smartphone, tablet, etc.) so as to display scatter plots and/or box plots of measured characteristics of the cell suspension. In some variations, the cell detection, measurement, and/or counting device 302 may include a touch screen to enable a user to change gating parameters, select assay options, change pre-defined threshold, a combination thereof, and/or the like.

In some variations, the system 300 may optionally include measuring devices 310 such as Eppendorf tubes, vials, test tubes, pipettes, a combination thereof, and/or the like. In some variations, the measuring devices 310 may be used to transfer the cell suspension to the cell detection, measurement, and/or counting device 302.

Exemplary Cell Suspension with High Therapeutic Potential

Forty-one exemplary cell suspensions were prepared and analyzed using the methods described herein. These cell suspensions were prepared according to protocol and considered to have therapeutic potential.

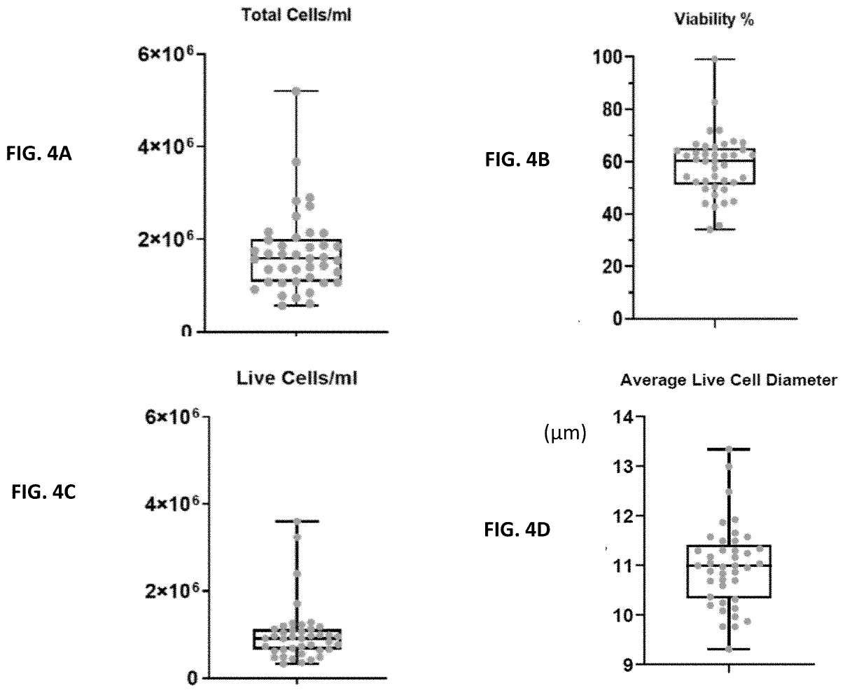

FIG. 4A illustrates a box plot of total number of cells (e.g., cell count) in the forty-one exemplary cell suspensions with high therapeutic potential. As seen in FIG. 4A, the box plot illustrates a minimum value of 567,000 cells/ml, a median value of 1,580,000 cells/ml, and a maximum value of 5,200,000 cells/ml. Among the analyzed cell suspensions, the 25th percentile value and 75th percentile for total cells were about 1,090,000 cells/ml and about 2,010,000 cells/ml, respectively. Based on the minimum value of total cell count in this data, in some variations, the minimum threshold value for cell count (e.g., total cell count including viable cells and non-viable cells per unit volume) to consider a cell suspension as having sufficient therapeutic potential may be 550,000 cells/ml. In other words, a cell suspension may be considered to have high therapeutic potential if the cell count exceeds 550,000 cells/ml.

FIG. 4B illustrates a box plot of cell viability in the exemplary cell suspensions with high therapeutic potential. As seen in FIG. 4B, the box plot illustrates a minimum value of 34.1%, a median value of 60.20%, and a maximum value of 99.1%. The 25th percentile and 75th percentile values for cell viability were about 51.2% and about 65.05%, respectively. Based on the minimum value of total cell count in this data, the minimum threshold value for cell viability to consider a cell suspension as having sufficient therapeutic potential may be 30%. In other words, a cell suspension may be considered to have high therapeutic potential if the cell viability exceeds 30%.

FIG. 4C illustrates a box plot of total number of viable cells (e.g., cell count) in the exemplary cell suspensions with high therapeutic potential. As seen in FIG. 4C, the box plot illustrates a minimum value of 328,000 cells/ml, a median value of 910,000 cells/ml, and a maximum value of 3,600,000 cells/ml. The 25th percentile and 75th percentile values for total number of viable cells were about 650,000 cells/ml and about 1,230,000 cells/ml, respectively. Based on the minimum value of viable cell count in this data, the minimum threshold value for viable cell count per unit volume to consider a cell suspension as having sufficient therapeutic potential may be 300,000 cells/ml. In other words, a cell suspension may be considered to have high therapeutic potential if the total number of viable cells exceed 300,000 cells/ml.

FIG. 4D illustrates a box plot of average (mean) cell size in the exemplary cell suspensions with high therapeutic potential. As seen in FIG. 4D, the box plot has a minimum value of 9.305 μm, a median value of 10.98 μm, and a maximum value of 13.34 μm. The 25th percentile and 75th percentile values for average cell size were about 10.34 μm and about 11.41 μm, respectively. Based on the minimum value of average cell size in this data, the minimum threshold value for average diameter of cells to consider a cell suspension as having sufficient therapeutic potential may be 9 μm. A cell suspension may be considered to have high therapeutic potential if the average cell size exceeds 9 μm. Since the minimum value of the box plot 9.305 μm exceeds the minimum threshold value, the cell suspension may be considered to have high therapeutic potential.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

What is claimed is:

1. A cell suspension, comprising:
a population of cells derived from freshly disaggregated tissue, comprising at least 550,000 total cells per milliliter, wherein the population of cells has at least 30% viability, wherein at least 300,000 cells are viable, and wherein the median live cell diameter of the viable cells is greater than or equal to 9 micrometers; and
an effective amount of buffer.

2. The cell suspension of claim 1, wherein the tissue comprises skin tissue.

3. The cell suspension of claim 2, wherein the population of cells comprises keratinocytes, melanocytes, and fibroblasts.

4. The cell suspension of claim 1, wherein the population of cells is obtained by a process comprising:
obtaining a split-thickness skin sample;
contacting the epidermal sample with an effective amount of warmed enzyme solution;
contacting the epidermal sample with an effective amount of buffer;
mechanically disaggregating the epidermal sample;
contacting the epidermal sample with a second effective amount of buffer to create a buffered disaggregated tissue solution; and
passing the buffered disaggregated tissue solution through a filter, wherein the screen is at least as narrow as 100 micrometers.

6. A cell suspension therapeutic, comprising:
a population of cells derived from freshly disaggregated tissue, comprising at least 550,000 total cells per milliliter, wherein the population of cells has at least 30% viability, wherein at least 300,000 cells are viable, and wherein the median live cell diameter of the viable cells is greater than or equal to 9 micrometers;
an effective amount of buffer; and
at least one scaffolding element.
